(19) United States
(12) Patent Application Publication
(10) Pub. No.: US 2017/0172655 A1
(43) Pub. Date: Jun. 22, 2017

(54) SYSTEM AND METHOD FOR TREATMENT OF BARRETT'S ESOPHAGUS INCORPORATING RADIOMETRIC SENSING

(71) Applicants: Steven D. SCHWAITZBERG, Portland, ME (US); Meridian Medical Systems, LLC, Portland, ME (US)

(72) Inventors: Robert Allison, Rancho Palos Verdes, CA (US); Steven D. Schwaitzberg, Buffalo, NY (US)

(21) Appl. No.: 15/127,059

(22) PCT Filed: Mar. 20, 2015
(86) PCT No.: PCT/US15/21755

§ 371 Date: Sep. 19, 2016

Related U.S. Application Data
(60) Provisional application No. 61/968,476, filed on Mar. 21, 2014.

(51) Int. Cl. A61B 18/18; A61B 18/20; A61B 18/02; A61B 18/12; A61B 5/01; A61B 5/05

(52) U.S. Cl. CPC A61B 18/1815; A61B 5/01; A61B 5/0507; A61B 18/02; A61B 18/12; A61B 18/20; A61B 18/04

(57) ABSTRACT

The invention provides, inter alia, devices and systems adapted for treating Barrett's esophagus and associated methods of treatment using the devices. In certain embodiments, the invention provides an ablation catheter and method of use to endoscopically access portions of the human esophagus where undesired growth of epithelium may develop. In some embodiments, the devices and systems include a microwave antenna, an ablation means (such as a microwave ablation means), an expandable basket configured to accommodate an optical inspection system proximate to the ablation means and a radiometer coupled to the antenna, the radiometer being configured to measure, e.g., temperature and or impedance mismatch from the antenna.

This application claims the benefit of U.S. Provisional Application No. 61/968,476, filed on Mar. 21, 2014.

The entire teachings of the above application(s) are incorporated herein by reference.

### BACKGROUND OF THE INVENTION

The clinical significance of Barrett's Esophagus is that it is a known precursor condition to adenocarcinoma of the esophagus, a particularly lethal cancer with rapidly increasing incidence. The condition involves the replacement of normal stratified squamous epithelium with simple columnar epithelium, which is the result of, e.g., chronic gastroesophageal reflux disease (GERD). Accordingly, a need exists for devices and systems suitable for ablating undesired tissue growth associated with, e.g., Barrett's esophagus, as well as associated methods of using them.

### SUMMARY OF THE INVENTION

The invention provides, inter alia, devices and systems adapted for treating Barrett's esophagus and associated methods of treatment using the devices and systems. In certain embodiments, the invention provides an ablation catheter and methods of using it to endoscopically access portions of the human esophagus where undesired growth of epithelium may develop. The ablation system is, in certain embodiments, a non-contact system able to perform an ablation that entails delivering energy to an entire circumference, or a portion thereof, in the esophagus. In some embodiments, the device or system employs, for the ablation, a microwave energy system, while other embodiments use RF (radiofrequency), ultraviolet (UV), ultrasound, laser, or cryoablation. A non-contact position of the catheter/probe is achieved, in some embodiments, through use of an expandable basket stand-off (also referred to as an expandable basket, herein). The system, incorporating this basket, allows visualization of the treatment target during a course of therapy. The system, incorporating radiometric sensing, monitors delivery of energy during ablation to ensure completeness of energy delivery and control therapy to enable a therapeutic goal of healthy tissue regeneration after elimination of abnormal tissue.

Advantageously, the devices, systems, and methods provided by the invention:
a) enable optical inspection (e.g., via endoscope) during an ablation procedure, unlike existing modalities incorporating balloons;
b) are “one size fits all”, eliminating the need for multiple catheter/probe balloons and eliminating the need to perform “balloon sizing/staging” prior to placement of catheter/probe and ablation;
c) recognize the diameter of an esophagus to be treated, and therefore deliver optimal energy to diseased tissue, while monitoring the heating profile during course of therapy; and
d) provide a single system that will treat the spectrum of patients. The system stand-off (expandable basket) will expand to fit to a range of esophageal diameters and the system will adapt sensing and energy delivery. This is a distinct advantage over existing systems, which require that different balloon sizes be employed to fit a specific esophagus size and ultimately cannot ensure equal or sufficient contact with all diseased tissue, which results in under-therapy and the need to conduct repeated therapies focusing on gaps in initial intervention.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 provides several schematic diagrams of an antenna and choke system useful in the present invention. The illustrated antenna is an omni-directional dipole antenna.

FIG. 2 provides several schematic diagrams of antenna and optional shield systems (which may be attached to an expandable basket, not shown) deployed in a tissue, such as an esophagus. Also shown are illustrative heating patterns that demonstrate the shield's skewing of the heating pattern.

FIG. 3 is a schematic diagram of an antenna, shield, and choke systems deployed in a tissue, such as an esophagus. The figure illustrates how the choke element significantly attenuates fold back of the microwave field.

FIG. 4 is a schematic diagram of circuitry, antenna, and basket elements deployed in an esophagus. The illustrative antenna is an omni-directional antenna. The basket is deployed to stretch the esophagus, e.g., to its maximum diameter, thereby creating an approximate circular geometry without folds. A centering device positions the antenna on the center line of the basket. A deployment means opens and closes the expandable basket. The basket allows for optical inspection of the treatment region with an optical inspection system. The impedance mismatch between the antenna and esophagus is a function of the esophagus diameter. In some embodiments, a reflectometer in either or both of the radiometer and generator functions at different frequencies to measure the impedance mismatch and determine the diameter of the esophagus. The optimum microwave power required for desired treatment is determined for the specific diameter and impedance mismatch. The radiometer monitors heating progress. For an omni-directional antenna, heating should be a substantially continuous ring. Other circuitries and antenna configurations can be adapted consonant with the present invention.

FIG. 5 is a schematic diagram of antenna and basket elements deployed in an esophagus. The illustrative antenna in this figure is a directional antenna that creates a non-uniform heating pattern in the esophagus.

FIG. 6 is a plot of mismatch measurements made using a radiometer useful in the present invention. This plot is of measured data showing the relationship between radiometer output voltage (in the reflection measurement mode) to reflection coefficient squared (Γ²). A simple straight line equation relates Γ² to output voltage. Γ² is, in turn, related to esophagus diameter.

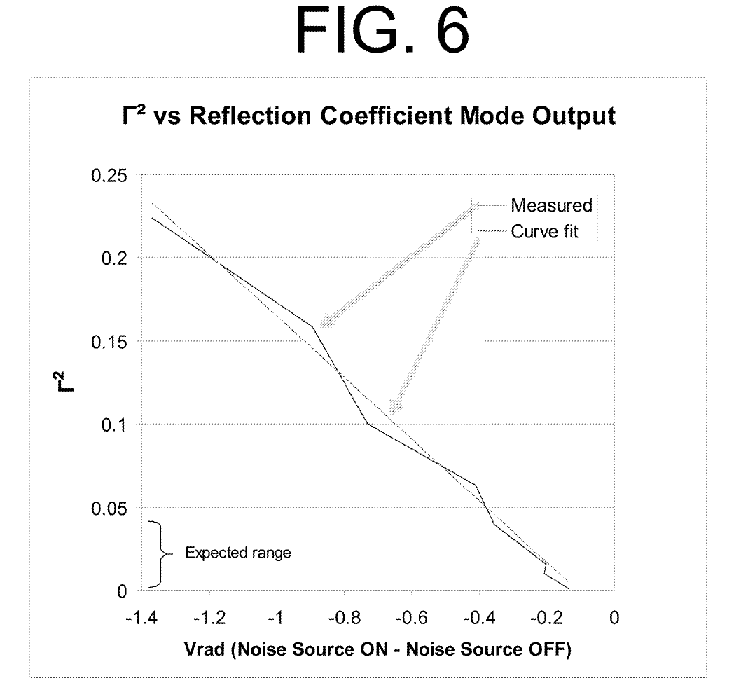

### DETAILED DESCRIPTION OF THE INVENTION

A description of example embodiments of the invention follows.

In one aspect the invention provides devices and systems that include an elongated probe that is substantially cylindrically shaped, where the probe includes a microwave antenna and an ablation means. In certain embodiments, the antenna and abaltion means may be separate, e.g., they are deployed in different probes. The probe can be located substantially centrally in an expandable basket that, in turn, is substantially cylindrically shaped, the probe and basket being configured to accommodate an optical inspection system (such as an endoscope) proximate to the ablation means. These devices and systems also include a radiometer coupled to the antenna, the radiometer being configured to measure temperature from the antenna, the radiometer further being coupled to a control of the ablation means. The antenna facilitates microwave radiometric sensing feedback to control delivery of energy, e.g., by measuring temperature of a tissue to be treated (or during treatment) as well as the impedance mismatch at a sensing frequency and, where the ablation means is by microwaves, optionally at a heating frequency (e.g., impedance mismatch can be measured at either or both the sensing and heating frequencies), e.g., between the antenna and esophagus wall (at the heating frequency) and/or between the radiometer and antenna (at the sensing frequency), which allows the system to approximate the esophageal diameter and therefore tune the ablation means to deliver an appropriate amount of energy. Therefore the devices and systems also comprise circuitry to measure impedance mismatch, the circuitry being coupled to a control of the abalation means. The devices and systems described herein are collectively “devices and systems provided by the invention” or “devices or systems provided by the invention.”

“Substantially cylindrically shaped” means a substantially circular z-axis (i.e., down the longest primary axis) projection with substantially constant circumference along the z-axis. In certain embodiments, “substantially constant circumference” means less than about: 50, 45, 40, 35, 30, 25, 20, 15, 10, or 5% deviation of the circumference of the z-axis projection over a length of the object (e.g., about: 10, 25, 50, 75, 80, 90, 95, or 100% of the z-axis length). For example, a biological surface, such as a portion of an esophagus or a portion of an intestine (or similar tissues), may include numerous tissue folds, but still has a substantially cylindrical shape. In the present invention, an expandable basket (also known as a stand-off) unfolds any tissue folds so that the microwave ablation energy is absorbed more or less uniformly around the periphery and no portion of the surface is in the shadow of a fold. When using the expandable basket, the cross section may be somewhat flattened or elliptical in shape rather that exactly circular.

“Coupled”, as in, e.g., the radiometer and/or circuitry to measure impedance mismatch are coupled to a control of the ablation means, refers to a communicative coupling that facilitates the effective transfer of electrical signals and encompasses wireless as well as physically wired connections, such as wires and cables, such as coaxial cables. A “control”, as in a control of the ablation means, is a circuitry that facilitates modulating the intensity and/or frequency of the ablation means. For example, a control of the ablation means (such as a microwave ablation means) can increase or decrease the frequency, intensity, or frequency and intensity of the energy emitted from the abalation means. Accordingly, a coupling of the radiometer, circuitry to measure impedance mismatch, or the radiometer and circuitry to measure impedance mismatch to a control of the abalation means produces a feedback system that effectively controls the ablation process. The controls of the abalation means, e.g., from the radiometer and circuitry to measure impedance mismatch, can be separate controls or a single control that integrates both signals.

“Optical inspection systems” include both still and video cameras that can operate in one or more of the visible, infrared, and ultraviolet frequencies. In particular embodiments, the optical inspection system is an endoscope. In certain embodiments, the expandable basket includes one or more windows that allow the optical inspection system to monitor the tissue being ablated in real time i.e., before, during, or after ablation.

An “ablation means” is a component that affects the ablation (destruction or removal) of undesired tissue. Exemplary ablation modalities include microwave, radiofrequency, ultrasound, laser, cryogenic ablation, or a combination thereof. In particular embodiments, the ablation means is microwave ablation e.g., the antenna of the device or system delivers microwave energy from a generator or transducer.

An “expandable basket” provides a substantially uniform spacing of a probe from, e.g., a tissue to be ablated. An expandable basket typically makes contact with the tissue to be ablated (and, in some embodiments, surrounding tissue), and can expand the tissue to, e.g., reduce or eliminate tissue folds and provide more uniform surface for ablation.

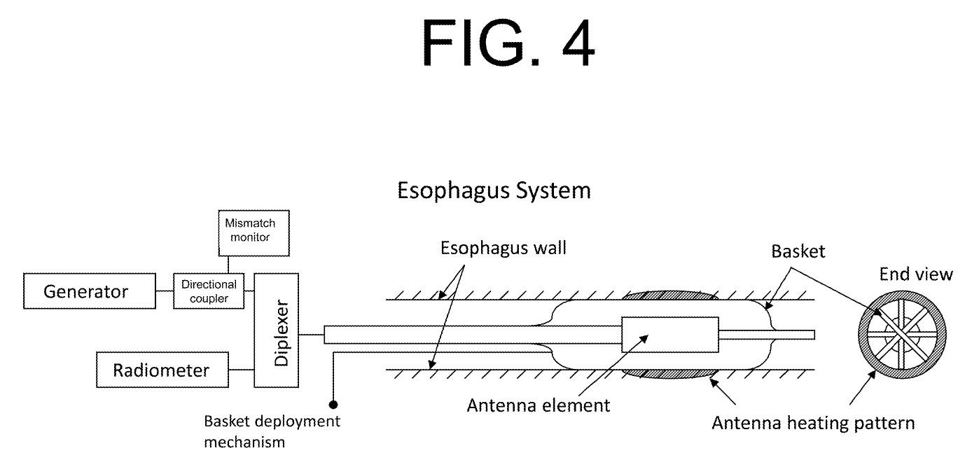

Any suitable radiometer can be used consonant with the invention. In particular embodiments, the radiometer is a Dicke radiometer. Exemplary radiometers are also described in U.S. Pat. Nos. 6,496,738 and 6,210,367, which are incorporated by reference in their entirety. The devices (such as radiometers) described herein and in the above patents can be configured on a single integrated circuit. Such integrated circuits can further comprise elements described in the devices provided by the invention, such as a generator or transducer of the ablation means, circuitry for measuring impedance mismatch, and additional elements, such as those illustrated in FIG. 4 or otherwise described herein.

Microwave frequencies are typically used for detection of temperature and impedance in the present invention (e.g., at a sensing frequency), including microwave energy from about 1 to about 30 GHz, e.g., about 1-5 GHz, more particularly, about 3-5 GHz, and, still more particularly, about 4 GHz, e.g., 4 GHz +/−200 MHz. In some embodiments, microwaves are the ablation means used for heating, e.g., at a heating frequency, where the heating frequency is different from the sensing frequency. A heating frequency can also be used for measuring an impedance mismatch. Exemplary heating frequencies, in particular embodiments, are about: 2.5 GHz (e.g. about 2.45 to about 2.55 GHz), 900 MHz (e.g., about 850 to about 950 MHz), or for shallower tissue heating, such as surface heating, about 10-20 GHz. Suitable frequencies can be selected by the skilled artisan based on well-understood principles, e.g., the scanning depth in a target sample, e.g., a tissue, and associated parameters, such as antenna size relative to the application (e.g., for use in an adult versus a neonate) on a body. Multiple frequencies can be used, e.g., both for sensing and, in certain embodiments, ablation. For example, to monitor temperature and/or impedance mismatch 1, 2, 3, 4, 5, or more different frequencies (or bandwidths) can be used. Similarly, where microwave or radio frequencies are used for ablation, 1, 2, 3, 4, 5, or more different frequencies (or bandwidths) can be used to apply energy.

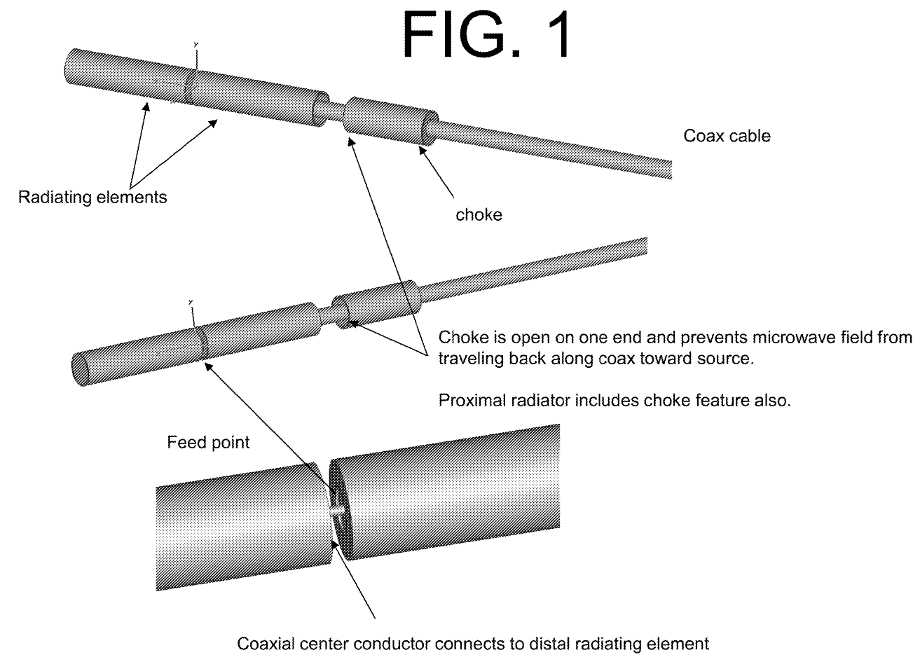

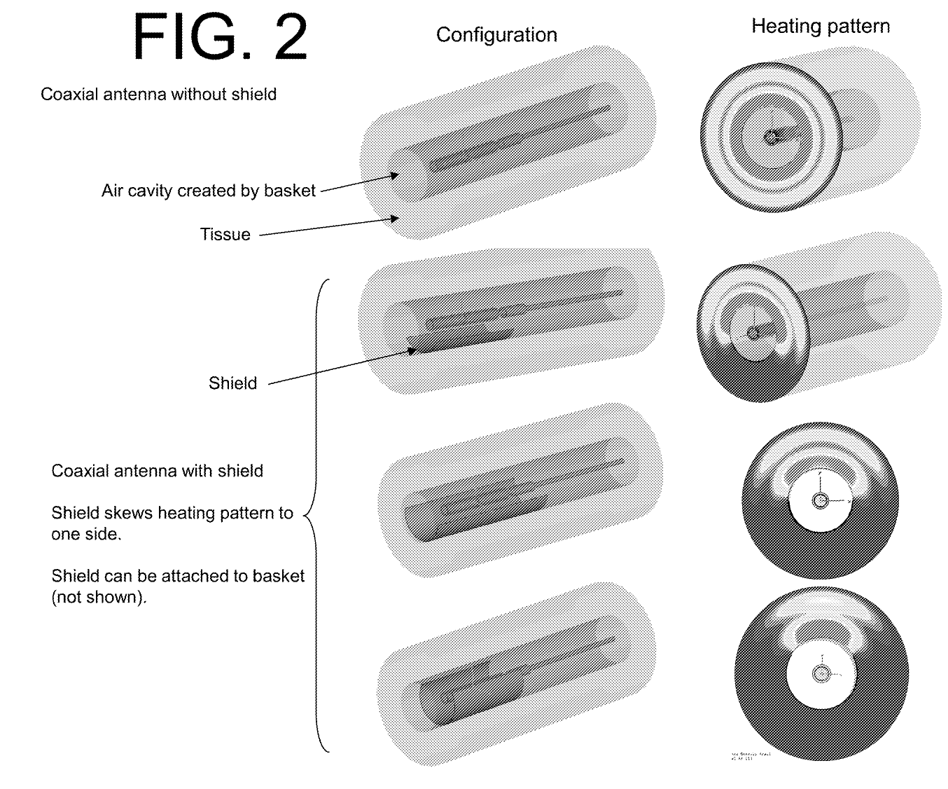

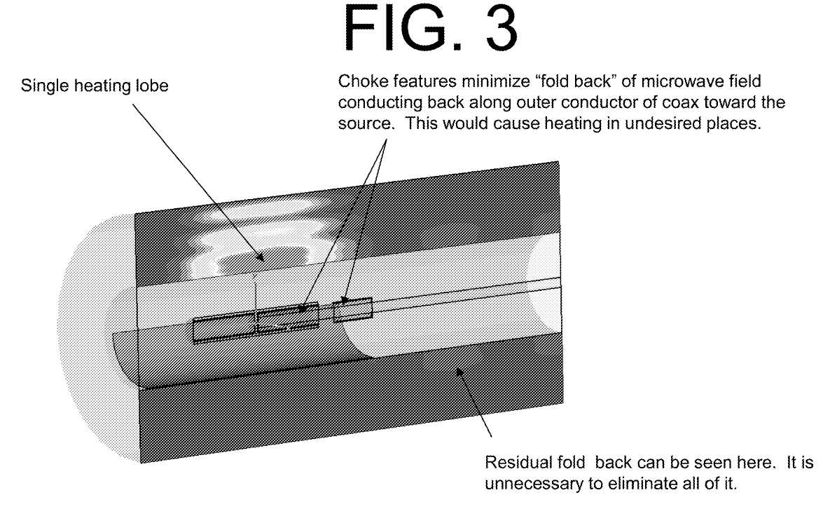

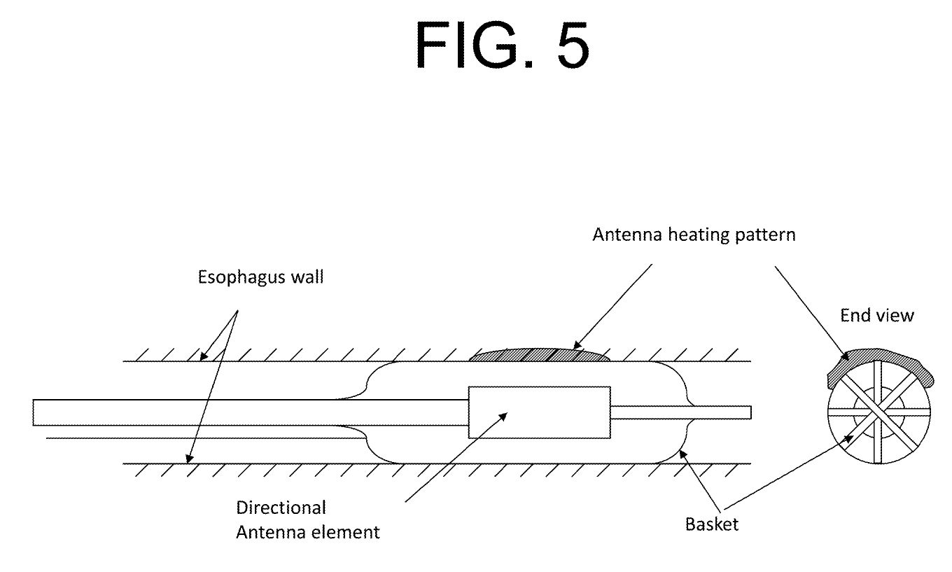

Any antenna suitable for use with the radiometer element of a device or system provided by the invention can be used and configured for the appropriate application. In certain embodiments, the antenna may be a disposable or an easily replaceable element. Antennas for use in the present invention are typically omni-directional or dipole type to facilitate heating and sensing from all around the circumference of a probe, except where a shield element is employed to sense and/or apply energy over less than the full circumference of the probe. FIGS. 1-5 show exemplary antenna configurations that can be used in the present invention—i.e., both with and without a shield element. In particular embodiments, the antenna is omni-directional but the device or system includes one or more shields partially surrounding the probe over an angle of a circle circumscribing the circumference of the probe, the one or more shields being adapted to at least partially attenuate (e.g., at least about: 50, 60, 70, 80, 90, 95, 96, 97, 98, 99, 99.9, 99.99% attenuation) the effect of the ablation means over the angle. In particular embodiments, the angle is between about 60 and about 300 degrees, e.g., about: 30, 40, 50, 60, 70, 80, 90, 100, 120, 140, 160, 180, 200, 220, 240, 260, 270, 280, 300, 310, 320, or 330 degrees. In these particular embodiments, the omni-directional antenna is converted into a directional antenna. In some embodiments, the expandable basket is made up of shield elements and, e.g., includes a window with an angle of about 30 to about 300 degrees to allow the ablation of tissue through the window. Shields suitable for the given abalation means can readily be identified by the skilled artisan and will act as conductors for the energy of the ablation means. Exemplary shield materials can include metals, such as metal foils.

A device or system provided by the invention includes circuitry configured to measure impedance mismatch. For example, circuitry configured to measure impedance mismatch can be configured to measure the magnitude of impedance mismatch. As used herein, “impedance mismatch” means the deviations between different impedance measurements (e.g., substantially concurrent measurements or in comparison to reference expected values), for example, by the radiometer and the antenna. Impedance can be measured by any means and refers to measurements of the relative permittivity (dielectric constant) and can include discreet or continuous measurements of magnitude (relative or absolute) and/or phase, as well as rates of change (e.g., first, second, or higher-order derivatives) or various transformations thereof. Exemplary transformations include time and/or space integrals or normalization (such as mean variance normalization) of any of these measurements (or transforms) to predetermined standards. Such predetermined standards can, e.g., be stored in a database, which may optionally be stored in a non-transient computer-readable medium in a device or system provided by the invention or in an external database communicatively coupled to a device or system provided by the invention. Impedance mismatch can be measured at a single frequency or over a bandwidth (e.g., at about: 2, 3, 4, 5, 6, 7, 8, 9, 10, 20, 50, 100, or more frequencies). Impedance mismatch is measured at the sensing frequency(ies) (e.g. when sensing temperature) or, for example, where microwaves are used as the ablation means, at the heating frequency(ies), or both the heating and sensing frequency(ies). In particular embodiments, the circuitry configured to measure impedance mismatch is configured to passively measure the magnitude of impedance mismatch. In particular embodiments, the circuitry configured to measure impedance comprises a directional coupler and a microwave source. For example, where the ablation means relies on microwave energy, the generator acts as the microwave signal source. Where ablation means other than microwave energy may be used, a separate microwave signal source could be used. An exemplary system configured to measure impedance mismatch is shown in FIG. 4.

In other embodiments, the circuitry configured to measure impedance mismatch is configured to actively measure the magnitude of impedance mismatch. In more particular embodiments, the circuitry configured to measure impedance mismatch further measures the phase of the impedance mismatch. In certain particular embodiments, the circuitry configured to measure impedance mismatch comprises a quadrature detection scheme. In more particular embodiments, the circuitry configured to measure impedance mismatch comprises a directional coupler, a microwave oscillator, and an I/Q demodulator. In certain particular embodiments, the circuitry configured to measure impedance mismatch comprises an amplitude modulator, such as a Dicke switch. In particular embodiments, for a device or system configured to actively measure impedance mismatch, the circuitry configured to measure impedance mismatch is configured to measure the complex impedance mismatch.

In some embodiments where the system is configured to measure impedance mismatch, the radiometer and circuitry configured to measure impedance mismatch are formed on a single integrated circuit.

In particular embodiments, the ablation means in a device or system provided by the invention is a microwave ablation means, and the system further includes a microwave transmitter coupled to the microwave antenna and a diplexer between the antenna and the microwave transmitter and radiometer. An example of this configuration is shown in FIG. 4.

In certain embodiments, the devices and systems provided by the invention include a choke element adapted to attenuate electromagnetic radiation travelling along the exterior of the coupling of the probe to the radiometer. A “choke” is an inductor that attenuates high frequency alternating currents. In particular embodiments, the attenuation is about: 50, 60, 70, 75, 80, 85, 90, 91, 92, 93, 94, 95, 96, 97, 98, or 99%, or more.

The probe and expandable basket in the devices and systems provided by the invention are dimensioned and configured to be inserted into an air-filled cavity, such as an air-filled biological tissue, such as an esophagus, small bowel, colon, stomach, or trachea and bronchial tree. In particular embodiments, the probe and expandable basket, when closed, can fit within an esophagus with a diameter of at least about 1.5 to about 2.0 cm (e.g., are less than about 1.5 to about 2.0 cm). In certain embodiments, the expanded basket can reach a fully expanded diameter of about 1.5 to about 3.5 cm (e.g., about: 15, 20, 25, 30, or 35 mm). In certain particular embodiments, the probe has a diameter of about: 2, 3, 4, 5, 6, 7, 8, 9, or 10 mm, or more, e.g., about: 11, 12, 13, 14, or 15 mm. In some embodiments, the probe and expandable basket, when closed, have a diameter of about: 6, 8, 10, 12, 14, or 15 mm. The devices and systems provided by the invention are also dimensioned and configured to be inserted into, e.g., an esophagus and to reach the lower esophageal sphincter and so are configured to be insertable up to about 10 to about 50 cm (e.g., about: 10, 15, 20, 25, 30, 35, 40, 45, or 50 cm). For example, in some embodiments, the probe and expandable basket are separated from the radiometer and other circuitry, e.g., by a coaxial cable that is about 10 to about 50 cm long, or more. The devices and systems provided by the invention can readily be dimensioned and configured to treat other air-filled biological tissues analogously to the esophagus.

In particular embodiments, the device or system provided by the invention further includes an optical inspection system (such as an endoscope) proximate to the ablation means.

In certain embodiments, a device or system provided by the invention includes an user-readable output. In more particular embodiments, the user-readable output provides a user-readable display of one or more of: a tissue temperature, a impedance mismatch, and an image of a biological tissue proximate to the probe.

The devices and systems provided by the invention can include suitable power adapters and can be connected to external power supplies. In certain embodiments, a device or system provided by the invention includes a closed power source, such as a battery, for use as a remote device.

In certain embodiments, a device or system provided by the invention includes a non-transient storage medium for recording the measured temperature and/or impedance mismatch. In some embodiments, a device or system provided by the invention includes a non-transient storage medium with reference values of temperature and/or impedance mismatch stored thereon, the reference values being suitable for measuring temperature and/or characterizing an air-filled biological tissue (such as an esophagus, small bowel, colon, stomach, or trachea and bronchial tree), by non-contact measurement from the lumen of the tissue, such as the tissue's diameter and state, including in a healthy, compressed (e.g., as a measure of device depth in the tissue), or diseased (e.g., plaqued/occluded (e.g., atherosclerotic), burnt, scarred, cirrhotic, or cancerous) state.

In certain embodiments, a device or system provided by the invention is in close proximity with the luminal surface of a substantially cylindrical air-filled biological tissue (such as an esophagus, small bowel, colon, stomach, or trachea and bronchial tree) e.g., the expandable basket is in contact with the tissue but the probe is not. In particular embodiments, the biological tissue is of a mammal, such as a human. In certain particular embodiments, the biological tissue is of an esophagus, which, in more particular embodiments, is diseased, e.g., in the form of Barrett's esophagus. The Barrett's esophagus can, in varying embodiments, be diagnosed and/or treated using a device or system provided by the invention. In particular embodiments, the Barrett's esophagus is treated and/or diagnosed in a subject with GERD.

Accordingly, in related aspects, the invention provides methods of non-contact ablation of the luminal surface of an air-filled biological tissue, such as an esophagus, small bowel, colon, stomach, or trachea and bronchial tree. These methods entail placing the system of any one of the preceding claims within the luminal surface of the biological tissue in need of ablation and applying the ablative means, thereby ablating the luminal surface of the biological tissue, where the probe is not in physical contact with the luminal surface of the biological tissue. In certain embodiments, the methods entail multiple applications of ablative energy, such as 2, 3, 4, 5, or more applications of ablative energy.

In another related aspect, the invention provides methods of treating Barrett's Esophagus. These methods entail placing a device or system provided by the invention within the luminal surface of the esophagus of a mammal in need of treatment and applying the ablative means, thereby treating the Barrett's esophagus. Typically, the probe is not in physical contact with the luminal surface of the esophagus. As described above, the invention encompasses methods of treating other disorders analogously to Barrett's esophagus, e.g., by placing a device or system provided by the invention proximate to the tissue to be treated and applying the ablative means. Collectively, these are the “methods provided by the invention.”

In particular embodiments, the mammal is a human.

In certain embodiments, the expandable basket of the device or system is expanded to contact the biological tissue (e.g., esophagus, small bowel, colon, stomach, or trachea and bronchial tree) and reduce any tissue folds.

Any of the methods provided by the invention can include steps of one or more of: measuring the temperature or impedance mismatch of the air-filled biological tissue, optionally further comprising recording, displaying, or comparing (e.g., to reference standards), or a combination thereof, the temperature or impedance mismatch of the tissue. In certain embodiments, the methods provided by the invention include a step of visualizing the biological tissue, before, during, or after ablation, or a combination thereof, with an optical inspection system, such as an endoscope.

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

Headings used in this application are for convenience only and do not affect the interpretation of this application.

Preferred features of each of the aspects provided by the invention are applicable to all of the other aspects of the invention mutatis mutandis and, without limitation, are exemplified by the dependent claims and also encompass combinations and permutations of individual features (e.g., elements, including numerical ranges and exemplary embodiments) of particular embodiments and aspects of the invention, including the working examples. For example, particular experimental parameters exemplified in the working examples can be adapted for use in the claimed invention piecemeal without departing from the invention. For example, for materials that are disclosed, while specific reference of each of the various individual and collective combinations and permutations of these compounds may not be explicitly disclosed, each is specifically contemplated and described herein. Thus, if a class of elements A, B, and C are disclosed as well as a class of elements D, E, and F and an example of a combination of elements A-D is disclosed, then, even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application, including elements of a composition of matter and steps of method of making or using the compositions.

The forgoing aspects of the invention, as recognized by the person having ordinary skill in the art following the teachings of the specification, can be claimed in any combination or permutation to the extent that they are novel and non-obvious over the prior art—thus, to the extent an element is described in one or more references known to the person having ordinary skill in the art, they may be excluded from the claimed invention by, inter alia, a negative proviso or disclaimer of the feature or combination of features.

It should be understood that the example embodiments described above may be implemented in many different ways. In some instances, the various methods and machines described herein may be implemented by a physical, virtual, or hybrid general purpose computer, or a computer network environment.

Embodiments or aspects thereof may be implemented in the form of hardware, firmware, or software. If implemented in software, the software may be stored on any non-transient computer-readable medium that is configured to enable a processor to load the software or subsets of instructions thereof. The processor then executes the instructions and is configured to operate or cause an apparatus to operate in a manner as described herein.

Further, firmware, software, routines, or instructions may be described herein as performing certain actions and/or functions of the data processors. However, it should be appreciated that such descriptions contained herein are merely for convenience and that such actions, in fact, result from computer devices, processors, controllers, or other devices executing the firmware, software, routines, instructions, etc.

It should also be understood that the schematics may include more or fewer elements, be arranged differently, or be represented differently. But it should further be understood that certain implementation may dictate that the schematic be implemented in a particular way.

While this invention has been particularly shown and described with references to example embodiments thereof, it will be understood by those skilled in the art that various changes in form and details may be made therein without departing from the scope of the invention encompassed by the appended claims.

1. A system, comprising:
a. an elongated probe that is substantially cylindrically shaped, the probe comprising a microwave antenna and an ablation means, the probe being located substantially centrally in an expandable basket that is substantially cylindrically shaped, the probe and basket being configured to accommodate an optical inspection system proximate to the ablation means;
b. a radiometer coupled to the antenna, the radiometer being configured to measure temperature from the antenna, the radiometer coupled to a control of the ablation means; and
c. circuitry configured to measure impedance mismatch, the circuitry coupled to a control of the ablation means, wherein the circuitry configured to measure impedance mismatch measures the magnitude of an impedance mismatch and the magnitude of the impedance mismatch.

2. The system of claim 1, wherein the probe and expandable basket, when closed, have a diameter dimensioned less than about 1.5 to about 2.0 cm and wherein the probe is configured to be insertable up to about 10 to about 50 cm into an air-filled vessel.

3. The system of claim 1, wherein the probe has a diameter of about: 2, 3, 4, 5, 6, 7, 8, 9, or 10 mm, or more.

4. The system of claim 1, wherein the circuitry configured to measure impedance mismatch is configured to passively measure the magnitude of the impedance mismatch.

5. The system of claim 4, wherein the circuitry configured to measure impedance mismatch comprises a directional coupler and a microwave source.

6. The system of claim 1, wherein the circuitry configured to measure impedance mismatch is configured to actively measure the magnitude of the impedance mismatch.

7. (canceled)

8. The system of claim 6, wherein the circuitry configured to measure impedance mismatch comprises a quadrature detection scheme.

9. The system of claim 8, wherein the circuitry configured to measure impedance mismatch comprises a directional coupler, a microwave oscillator, and an I/Q demodulator.

10. The system of claim 6, wherein the circuitry configured to measure impedance mismatch comprises an amplitude modulator.

11. The system of claim 10, wherein the amplitude modulator is a Dicke switch.

12. The system of claim 1, wherein the circuitry configured to measure impedance mismatch is configured to measure the complex impedance mismatch.

13. (canceled)

14. The system of claim 1, wherein the ablation means is a microwave ablation means, the system further comprising a microwave transmitter coupled to the microwave antenna and a diplexer between the antenna and the microwave transmitter and radiometer.

15. The system of claim 1, wherein the antenna is omnidirectional.

16. The system of claim 1, wherein the antenna is directional.

17. The system of claim 16, further comprising one or more shields partially surrounding the probe over an angle of a circle circumscribing the probe, the one or more shields being adapted to at least partially attenuate the effect of the ablation means over the angle.

18. The system of claim 17, wherein the angle is between about 60 and about 300 degrees.

19. The system of claim 1, further comprising a choke element adapted to attenuate electromagnetic radiation travelling along the exterior of the coupling between the probe and the radiometer.

20. The system of claim 1, wherein the system further comprises an optical inspection system proximate to the ablation means.

21. (canceled)

22. (canceled)

23. (canceled)

24. (canceled)

25. The system of claim 1, wherein the device further comprises a non-transient storage medium with reference values of temperature and/or impedance mismatch stored thereon, the reference values being suitable for measuring temperature and/or characterizing an air-filled biological tissue, by non-contact measurement from the lumen of the air-filled biological tissue.

26. (canceled)

27. (canceled)

28. (canceled)

29. The system of claim 25, wherein the air-filled biological tissue is of an esophagus.

30. (canceled)

31. (canceled)

32. (canceled)

33. (canceled)

34. (canceled)

35. (canceled)

36. (canceled)

37. (canceled)

38. (canceled)
